(19) United States
(12) Patent Application Publication
(10) Pub. No.: US 2024/0104731 A1
(43) Pub. Date: Mar. 28, 2024

(54) System for Integrated Analysis of Multi-Spectral Imaging and Optical Coherence Tomography Imaging

(71) Applicant: Alcon Inc., Fribourg (CH)

(72) Inventors: Vignesh Suresh, Arlington, TX (US); Lu Yin, Keller, TX (US); Ramesh Sarangapani, Coppell, TX (US)

(21) Appl. No.: 18/475,387

(22) Filed: Sep. 27, 2023

Related U.S. Application Data
(60) Provisional application No. 63/377,300, filed on Sep. 27, 2022.

(51) Int. Cl. G06T 7/00; A61B 3/14; G06V 10/48; G16H 30/40

(52) U.S. Cl. CPC G06T 7/0012; A61B 3/14; G06V 10/48; G16H 30/40; G06T 2207/10101; G06T 2207/20081; G06T 2207/20084; G06T 2207/30041; G06V 2201/03

(57) ABSTRACT

In certain embodiments, a system, a computer-implemented method, and computer-readable medium are disclosed for performing integrated analysis of MSI and OCT images to diagnose eye disorders. MSI and OCT are processed using separate input machine learning models to create input feature maps that are input to an intermediate machine learning model. The intermediate machine learning model processes the input feature maps and outputs a final feature map that is processed by one or more output machine learning models that output one or more estimated representations of a pathology of the eye of the patient.

### BACKGROUND

Multispectral imaging (MSI) is a technique that involves measuring (or capturing) light from samples (e.g., eye tissues/structures) at different wavelengths or spectral bands across the electromagnetic spectrum. MSI may capture more information from the samples that may not be visible through conventional imaging, which generally uses broadband illumination and a broadband imaging sensor. The MSI information obtained by an MSI imaging system may be used to diagnose eye disorders and to enable real-time adjustment in the use of instruments (e.g., forceps, lasers, probes, etc.) used to manipulate eye tissues/structures during surgery.

Optical coherence tomography (OCT) is a technique that uses light waves to generate two dimensional (2D) and three-dimensional (3D) images of the eye. 2D OCT may involve the use of time-domain OCT and/or Fourier-domain OCT, the latter involving the use of spectral-domain OCT and swept-source OCT methods. 3D OCT may similarly utilize time-domain OCT and Fourier-domain OCT imaging techniques. OCT imaging may likewise be used pre-operatively to diagnose eye disorders or intra-operatively.

It would be an advancement in the art to better utilize the capabilities of MSI and OCT to diagnose eye disorders.

### SUMMARY

In certain embodiments, a system is provided. The system includes one or more processing devices and one or more memory devices coupled to the one or more processing devices. The one or more memory devices store executable code that, when executed by the one or more processing devices, causes the one or more processing devices to, for each imaging modality of a plurality of imaging modalities, process one or more images according to each imaging modality using an input machine learning model of a plurality of input machine learning models corresponding to each imaging modality to obtain an input feature map, the one or more images being images of an eye of a patient. The system processes the feature maps for the plurality of imaging modalities using an intermediate machine learning model to obtain a final feature map. The final feature map is processed using one or more output machine learning models to obtain one or more estimated representations of a pathology of the eye of the patient.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example system for performing integrated analysis of MSI and OCT images to diagnose eye disorders in accordance with certain embodiments.

FIG. 2A is diagram illustrating a first approach for training machine learning models to perform integrated analysis of MSI and OCT images to diagnose eye disorders in accordance to certain embodiments.

FIG. 2B is diagram illustrating a second approach for training machine learning models to perform integrated analysis of MSI and OCT images to diagnose eye disorders in accordance to certain embodiments.

FIG. 2C is diagram illustrating a third approach for training machine learning models to perform integrated analysis of MSI and OCT images to diagnose eye disorders in accordance to certain embodiments.

FIG. 3 is a flow diagram of a method for training machine learning models to perform integrated analysis of MSI and OCT images to diagnose eye disorders in accordance to certain embodiments.

FIG. 4 illustrates an example computing device that implements, at least partly, one or more functionalities for performing integrated analysis of MSI and OCT images in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Various embodiments described herein provide a framework for processing the information obtained from MSI and OCT images using artificial intelligence. An advantage of MSI is that MSI images contain rich information about the retina within the wide range of spectral bands and these are features that cannot be seen using human vision or a Fundus camera. The wide range of spectral bands of MSI further provides a high degree of depth penetration into the retina. However, an MSI image does not provide structural information. In contrast, OCT images do provide structural information about the retina. However, a high degree of expertise is required to interpret OCT images. Using the approach described herein, the rich detail and high depth penetration of MSI can be combined with the structural information of OCT to identify biomarkers for various pathologies and perform early disease diagnosis.

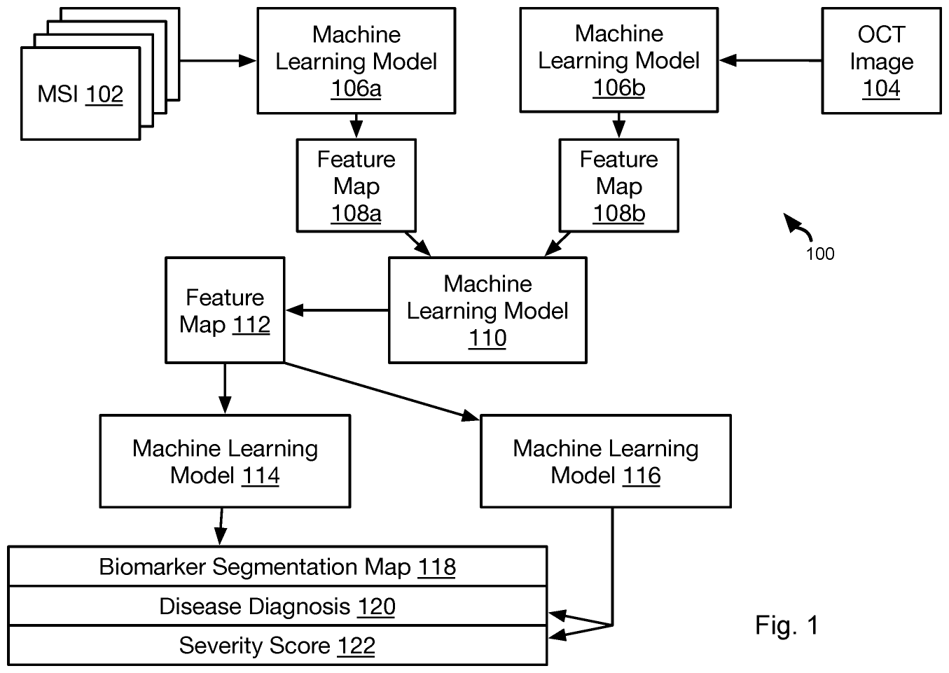

FIG. 1 illustrates a system 100 for performing integrated analysis of MSI images 102 and an OCT image 104. The system 100 may include three main stages, a feature extraction stage using machine learning models 106a, 106b, a feature boosting stage using machine learning model 110, and a biomarker and prediction stage using machine learning models 114, 116. Through those three stages, the system 100 processes MSI images 102 and OCT images 104 separately for feature extraction and then combines the extracted features to obtain meaningful interpretations.

The MSI images 102 may be captured using any approach for implementing MSI known in the art, including so-called hyper-spectral imaging (HSI). Likewise, the OCT image 104 may be obtained using any approach for performing OCT known in the art.

The MSI images 102 are obtained by illuminating the eye of a patient using multi-spectral band illumination sources (e.g., narrowband illumination sources, narrowband filters, etc.) and/or measuring reflected light using multi-spectral band cameras (e.g., an imaging sensor capable of sensing multiple spectral bands, beyond red, green, and blue (RGB) spectral bands). Accordingly, each MSI image 102 represents reflected light within a specific spectral ban. Differences among the MSI images 102 result from different reflectivities of different structures within the eye for different spectral bands. The MSI images 102, when considered collectively, therefore provide additional information about the structures of the eye than a single broadband image. In some implementations, the MSI images 102 are en face images of the retina that are used to detect pathologies of the retina. However, MSI images 102 of other parts of the eye, such as the vitreous or anterior chamber may also be used.

Optical coherence tomography (OCT) is a technique that uses light waves from a coherent light source, i.e., laser, to generate two-dimensional (2D) and three-dimensional (3D) images of the eye. OCT images are typically cross-sectional images of the eye for planes parallel to and colinear with the optical axis of the eye. However, OCT images for a plurality of section planes may be used to construct a 3D image, from which 2D images may be generated for section planes that are not parallel to the optical axis. For example, an en face image of the retina may be derived from the 3D image. In some embodiments, the OCT image 104 is such an en face image of the retina. OCT is capable of imaging the retina up to a certain depth such that the OCT image 104, in some embodiments, is a collection of en face images for image planes at or above the surface of the retina down to a depth within or below the retina.

Although the examples described herein relate to the use of MSI images 102 and OCT images 104, images from any pair of imaging modalities, or images from three or more different imaging modalities, may be used in a like manner. For example, additional imaging modalities may include scanning laser ophthalmology (SLO), a fundus camera, and/or a broadband visible light camera.

In the system 100, the MSI images 102 are processed by a machine learning model 106a and the OCT image 104 is processed by a machine learning model 106b. The machine learning models 106a, 106b may be implemented as a neural network, deep neural network (DNN), convolution neural network (CNN), recurrent neural network (RNN), region-based CNN (R-CNN), autoencoder (AE) or other type of neural network.

The result of processing the images 102, 104 by the machine learning models 106a, 106b are feature maps 108a, 108b, respectively. For example, the feature maps 108a, 108b may be the outputs of one or more hidden layers of the machine learning models 106a, 106b. The feature maps 108a, 108b may be two-dimensional or three-dimensional arrays of values. Where the feature maps 108a, 108b are two-dimensional arrays, the feature maps 108a, 108b may include identical sizes in both dimensions or may be different. Where one or both of the feature maps 108a, 108b is a three-dimensional array, the feature maps 108a, 108b may include identical sizes in at least two dimensions or may be different in any of the three dimensions.

The feature maps 108a, 108b, and possibly the images 102, 104, are processed by a machine learning model 110. The machine learning model 110 may be implemented as a neural network, deep neural network (DNN), convolution neural network (CNN), recurrent neural network (RNN), region-based CNN (R-CNN), autoencoder (AE) or other type of neural network. The result of processing the feature maps 108a, 108b, and possibly the images 102, 104, by the machine learning model 110 is a feature map 112. For example, the feature map 112 may be the outputs of one or more hidden layers of the machine learning model 110 as discussed in greater detail below.

The feature map 112, and possibly the images 102, 104, are then processed by a machine learning model 114 and a machine learning model 116, which then outputs one or more biometric segmentation maps 118, which label features of the eye represented in the images 102, 104 corresponding to one or more pathologies. Each biometric segmentation map 118 may be in the form of an image having the same size as the images 102, 104 and in which non-zero pixels correspond to pixels in the images 102, 104 identified as corresponding to a particular pathology represented by the biometric segmentation map. The biometric segmentation maps 118 may include a separate map for each pathology of a plurality of pathologies or a single map in which all pixels representing any of the plurality of pathologies are non-zero.

The machine learning model 114 may be implemented as a neural network, deep neural network (DNN), convolution neural network (CNN), recurrent neural network (RNN), region-based CNN (R-CNN), autoencoder (AE) or other type of neural network. For example, the machine learning model 114 may be implemented as a U-net.

The machine learning model 116 outputs a disease diagnosis 120 and possibly a severity score 122 corresponding to the disease diagnosis. The machine learning model 116 may be implemented as a long short term memory (LSTM) machine learning model, generative adversarial network (GAN) machine learning model, or other type of machine learning model. The disease diagnosis 120 may be output in the form of text naming the pathology, a numerical code corresponding to the pathology, or some other representation. The severity score 122 may be a numerical value, such as a value from 1 to 10 or a value in some other range. The severity score 122 may be limited to a discrete set of values (e.g., integers from 1 to 10) or may be any value within the limits of precision for the number of bits used to represent the severity score 122.

Pathologies for which biometric segmentation maps 118 may be generated and for which a diagnosis 120 and severity score 122 may be generated include at least those which cause perceptible changes to the retina, such as at least the following:
Retinal tears
Retinal detachment
Diabetic retinopathy
Hypertensive retinopathy
Sickle cell retinopathy
Central retinal vein occlusion
Epiretinal membrane
Macular holes
Macular degeneration (including age-related Macular Degeneration)
Retinal pigmentosa
Glaucoma
Alzheimer's disease
Parkinson's disease

The biometric segmentation maps 118 may, for example, mark vascular features that corresponding to a pathology. Examples of vascular features that can be used to diagnose a pathology are described in the following references, both of which are incorporated herein by reference in their entirety:
Segmenting Retinal Vessels Using a Shallow Segmentation Network to Aid Ophthalmic Analysis, M. Arsalan et al., Mathematics 2022, Volume 10, p. 1536.
PVBM: A Python Vasculature Biomarker Toolbox Based on Retinal Blood Vessel Segmentation, J. Fhima et al., Cornell University (31 Jul., 2022).

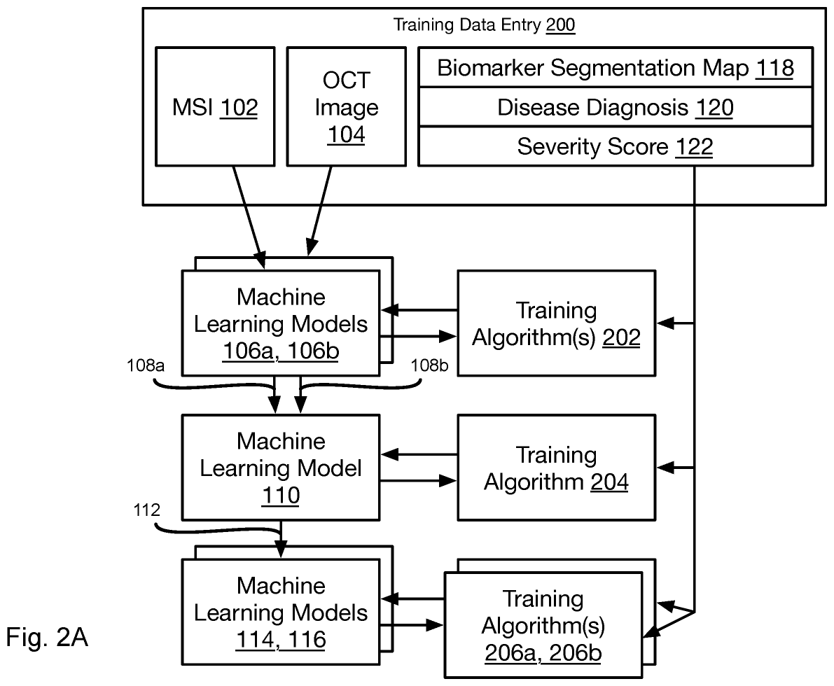

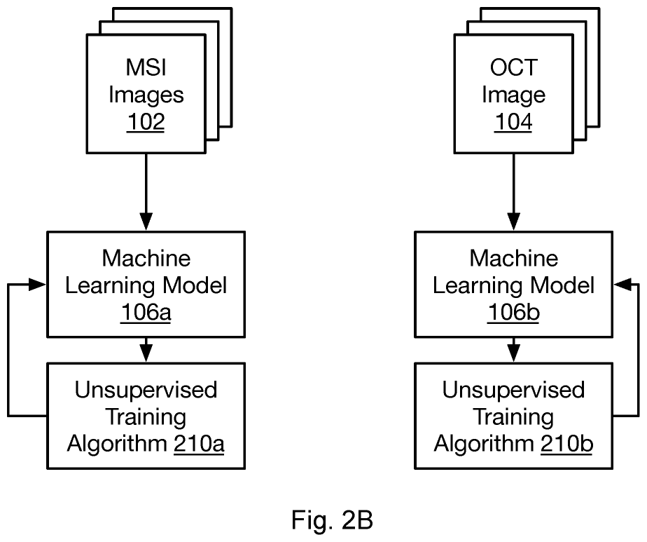

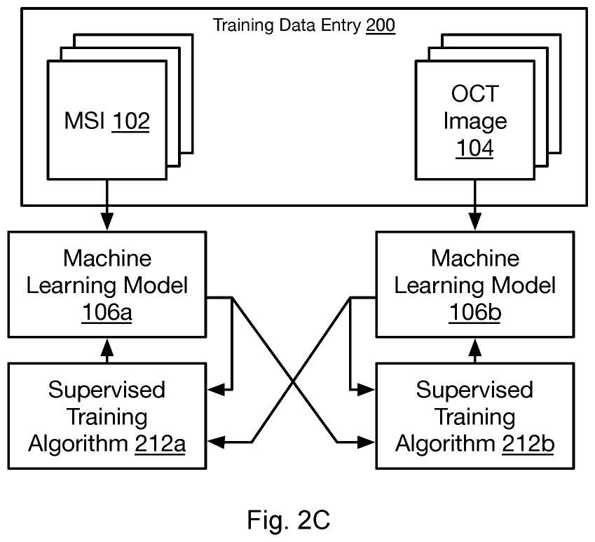

FIG. 2A illustrates an example approach for training the machine learning models 106a, 106b, 110, 114, 116. In particular, FIG. 2A illustrates a supervised machine learning approach that uses a plurality of training data entries 200, such as many hundreds, thousands, tens of thousands, hundreds of thousands, or more. Each training data entry 200 may include, as inputs, MSI images 102 and an OCT image 104. Each image of the MSI images 102 represents an image obtained by detecting light in a different spectral band relative to the other MSI images 102.

The MSI images 102 and OCT image 104 images of a training data entry 200 may be of the same eye of a patient and may be captured substantially simultaneously such that the anatomy represented in the images 102, 104 is substantially the same. For example, “substantially simultaneously” may mean within 1 second and 1 hour of one another. However, “substantially simultaneously” may depend on the pathologies being detected: those that have a very slow progression may use images 102, 104 with longer differences in times of capture, such as less than one day, less than a week, or some other time difference. The MSI images 102 and OCT image 104 are preferably aligned and scaled relative to one another such that a given pixel coordinate in the MSI images 102 represents substantially the same location (e.g., within 0.1 mm, within 1 μm, or within 0.01 μm) in the eye as the same pixel coordinate in the OCT image 104. This alignment and scaling may be achieved for the entire images 102, 104 or for at least a portion of one or both of the images 102, 104 showing anatomy of interest (e.g., the macula of the retina).

Alignment and scaling of the images 102, 104 relative to one another may be achieved by alignment of optical axes of instruments used to capture the images 102, 104 and calibrating the magnification of the instruments to achieve substantially identical scaling (e.g., within +/−0.1%, within 0.01%, or within 0.001%). Alternatively, alignment and scaling of the images 102, 104 may be achieved by analyzing anatomy represented in the images 102, 104. For example, where the MSI images 102 and OCT image 104 represent the retina of the eye, the pattern of blood vessels represented in each image 102, 104 may be used to align and scale one or both of the images 102, 104. Where the images 102, 104 are different sizes or do not completely overlap, such as after registering and scaling, non-overlapping portions of one or both of the images 102, 104 may be trimmed and/or one or both of the images 102, 104 may be padded such that the images 102, 104 are the same size and completely overlap one another.

Each training data entry 200 may include, as desired outputs, some or all of one or more biomarker segmentation maps 118, a disease diagnosis 120, and a severity score 122. A same patient may have multiple pathologies present such that a segmentation map 118, a disease diagnosis 120, and a severity score 122 may be included for each pathology present or a subset of most dominant pathologies. The desired outputs are generated by a human expert based on evaluations of the images 102, 104 and possibly other health information for the patient obtained before or after capture of the images 102, 104. In particular, the biomarker segmentation map 118 for a pathology may include pixels of one or both of the images 102, 104 marked by a human expert as corresponding to the pathology.

For each training data entry 200, the machine learning model 106a receives the MSI images 102 to produce one or more estimated biomarker segmentation maps. For example, the output of the machine learning model 106a may be a three-dimensional array in which each two-dimensional array along a third dimension is an estimated biometric segmentation map corresponding to a pathology.

A training algorithm 202 compares the one or more estimated biomarker segmentation maps to the one or more biomarker segmentation maps 118 for the training data entry 200. The training algorithm 202 then updates one or more parameters of the machine learning model 106a according to differences between each estimated biomarker segmentation map for a pathology and the corresponding biomarker segmentation map 118 for that pathology in the training data entry 200.

The machine learning model 106b may be trained in a like manner to the machine learning model 106a. For each training data entry 200, the machine learning model 106b receives the OCT image 104 and produces one or more estimated biomarker segmentation maps. For example, the output of the machine learning model 106a may be a three-dimensional array in which each two-dimensional array along a third dimension is an estimated biometric segmentation map corresponding to a pathology.

A training algorithm 202, which may be the same as or different from that used to train the machine learning model 106a, compares the one or more estimated biomarker segmentation maps to the one or more biomarker segmentation maps 118 of the training data entry 200. The training algorithm 202 then updates one or more parameters of the machine learning model 106b according to differences between each estimated biomarker segmentation map for a pathology and the corresponding biomarker segmentation map 118 for that pathology in the training data entry 200.

Where three or more imaging modalities are used, additional machine learning models may be present and trained in a like manner. Each machine learning model corresponds to an imaging modality and processes a corresponding image for that imaging modality in the training data entry. The machine learning model produces one or more estimated biomarker segmentation maps that are compared to the one or more biomarker segmentation maps 118 of the training data entry by a training algorithm, which then updates the machine learning model according to the comparison. A hidden layer for each machine learning model may produce outputs that are used as a feature map for the imaging modality to which the machine learning model corresponds.

As described above with respect to FIG. 1, the machine learning model 110 takes as inputs the feature maps 108a, 108b of the machine learning models 106a, 106b. The machine learning model 110 may be trained after the machine learning models 106a, 106b are trained with some or all of the training data entries 200.

For each training data entry 200, the machine learning model 110 receives feature maps 108a, 108b obtained from processing the MSI images 102 and OCT image 104 of the training data entry 200 with the machine learning models 106a, 106b. As noted above, the feature maps 108a, 108b may be the outputs of hidden layers of the machine learning models 106a, 106b, respectively, i.e., a layer other than the final layer that outputs the one or more estimated biomarker segmentation maps. The machine learning model 110 may also receive the MSI images 102 and OCT image 104 as inputs, though in other embodiments, only the feature maps 108a, 108b are used.

The machine learning model 110 processes the feature maps 108a, 108b, and possibly the MSI images 102 and OCT image 104, and produces one or more estimated biomarker segmentation maps. For example, the output of the machine learning model 110 may be a three-dimensional array in which each two-dimensional array along a third dimension is an estimated biometric segmentation map corresponding to a pathology. Although two feature maps 108a, 108b are shown, the machine learning model 110 may process any number of feature maps, and possibly any number of images used to generate the feature maps, in a like manner for any number of imaging modalities.

A training algorithm 204 compares the one or more estimated biomarker segmentation maps to the one or more biomarker segmentation maps 118 of the training data entry 200. The training algorithm 204 then updates one or more parameters of the machine learning model 110 according to differences between each estimated biomarker segmentation map for a pathology and the corresponding biomarker segmentation map 118 for that pathology.

As described above with respect to FIG. 1, the machine learning models 114, 116 takes as inputs the feature map 112 of the machine learning models 110. The machine learning models 114, 116 may be trained after the machine learning model 110 is trained with some or all of the training data entries 200.

For each training data entry 200, the machine learning models 114, 116 receive the feature maps 112 obtained from processing the MSI images 102 and OCT image 104 of the training data entry 200 with the machine learning models 106a, 106b, 110. As noted above, the feature map 112 may be the outputs of a hidden layer of the machine learning models 110, i.e., a layer other than the final layer that outputs the one or more estimated biomarker segmentation maps. The machine learning models 114, 116 may also take as inputs the MSI images 102 and OCT image 104, though in other embodiments, only the feature map 112 is used.

The machine learning model 114 processes the feature map 112, and possibly images 102, 104 from the training data entry 200, and produces one or more estimated biomarker segmentation maps. Where three or more imaging modalities are used, images according to the three or more imaging modalities from the training data entry 200 may be processed by the machine learning model 114 along with the feature map 112 obtained from the images. The output of the machine learning model 114 may be a three-dimensional array in which each two-dimensional array along a third dimension is an estimated biometric segmentation map corresponding to a pathology.

A training algorithm 206a compares the one or more estimated biomarker segmentation maps to the one or more biomarker segmentation maps 118 of the training data entry 200. The training algorithm 206a then updates one or more parameters of the machine learning model 114 according to differences between each estimated biomarker segmentation map for a pathology and the corresponding biomarker segmentation map 118 for that pathology.

The machine learning model 116 processes the feature map 112, and possibly the MSI images 102 and OCT image 104, and produces one or more estimated diagnoses and an estimated severity score for each estimated diagnosis. Where three or more imaging modalities are used, images according to the three or more imaging modalities from the training data entry 200 may be processed by the machine learning model 116 along with the feature map 112 obtained for the images.

The output of the machine learning model 116 may be a vector, in which each element of the vector, if nonzero, indicates a pathology is estimated to be present. The output of the machine learning model 116 may also be text enumerating one or more dominant pathologies estimated to be present. The output of the machine learning model 116 may further include a severity score for each pathology estimated to be present, such as a vector in which each element corresponds to a pathology and a value for an element indicates the severity of the corresponding pathology.

A training algorithm 206b compares the estimated diagnoses and corresponding severity scores to the disease diagnoses 120 and severity score 122 of the training data entry 200. The training algorithm 206b then updates one or more parameters of the machine learning model 116 according to differences between the estimated diagnoses and corresponding severity scores and the disease diagnoses 120 and severity score 122 of the training data entry 200.

Referring to FIG. 2B, in some embodiments, training of one or both of the machine learning models 106a, 106b may be performed by an unsupervised training algorithm 210a, 210b respectively. FIG. 2B shows training with images 102, 104 with the understanding that one or more machine learning models for additional or alternative imaging modalities can be trained in the same manner.

For the embodiment of FIG. 2B, the machine learning models 110, 114, 116 may be as described above with respect to FIG. 2A. In some embodiments, only one of the machine learning models 106a, 106b is trained using an unsupervised training algorithm 210a, 210b whereas the other is rained using a supervised training algorithm 202 as described above with respect to FIG. 2A. For the unsupervised machine learning algorithms 210a, 210b, labeled training data entries are not used.

The machine learning model 106a may be trained using a corpus of sets of MSI images 102. The corpus may be curated to include a large number of sets of MSI images, e.g., retinal images, of healthy eyes without pathologies present and a small fraction, e.g., less than 5 percent or less than 1 percent of the corpus, corresponding to one or more pathologies. The sets of MSI images 102 may or may not be labeled as to whether the set of images 102 represent a pathology and/or the specific pathology represented.

The unsupervised training algorithm 210a processes the corpus using the machine learning model 106a and trains the machine learning model 106a to identify and classify anomalies detected in the sets of MSI images 102 of the corpus. The unsupervised training algorithm 210a may be implemented using any approach for performing anomaly detection or other unsupervised machine learning known in the art. The output of the machine learning model 106a may be an image having the same dimensions as an individual MSI image 102 with pixels representing anomalies being labeled.

The machine learning model 106b may be trained using a corpus of OCT images 104. The corpus may be curated to include a large number of OCT images, e.g., retinal images, of healthy eyes without pathologies present and a small fraction, e.g., less than 5 percent or less than 1 percent of the corpus, corresponding to one or more pathologies. The OCT images 104 may or may not be labeled as to whether the set of images 102 represent a pathology and/or the specific pathology represented.

The unsupervised training algorithm 210b processes the corpus using the machine learning model 106b and trains the machine learning model 106a to identify and classify anomalies detected in the OCT images 104 of the corpus. The unsupervised training algorithm 210b may be implemented using any approach for performing anomaly detection or other unsupervised machine learning known in the art. The output of the machine learning model 106b may be an image having the same dimensions as each OCT image 104 with pixels representing anomalies being labeled.

The sets of MSI images 102 and OCT images 104 used to train the machine learning models by unsupervised training algorithms 210a, 210b may include images 102, 104 from the training data entries 200 used to train the other machine learning models 110, 114, 116. The sets of MSI images 102 and OCT images 104 may further be augmented with images of healthy eyes to facilitate the identification of anomalies corresponding to pathologies. The sets of MSI images 102 and OCT images 104 may be constrained to be the same size and may be aligned with one another. For example, although images 102, 104 are of a plurality of different eyes, the images 102, 104 may be aligned to place a representation of a center of the fovea of the retina at substantially the center of each image 102, 104, e.g., within 1, 2, or 3 pixels. Some other feature may be used for alignment, such as the fundus. Although images 102, 104 are of a plurality of different eyes, the images 102, 104 may also be scaled such that anatomy represented in the images is substantially the same size. For example, images 102, 104 may be scaled such that the fovea, fundus, or one or more other anatomical features are the same size.

Once trained, the machine learning models 106a, 106b may provide outputs to the machine learning model 110 (see FIGS. 1 and 2A) in the form of one or both of feature maps 108a, 108b that are the outputs of one or more hidden layers of the machine learning models 106a, 106b, respectively. Alternatively, the final outputs of the machine learning models 106a, 106b, e.g., images with anomaly labels, may be used as the inputs to the machine learning model 110.

Referring to FIG. 2C, in a refinement to the unsupervised machine learning approach of FIG. 2B, a supervised training algorithm 212b may compare the output of the machine learning model 106a to the output of the machine learning model 106b for a given set of MSI images 102 and an OCT image 104 of the same patient eye captured substantially simultaneously as defined above. The supervised training algorithm 212b may then adjust parameters of the machine learning model 106b according to the comparison in order to train the machine learning model 106b to identify the same anomalies detected by the machine learning model 106a. Note that the opposite approach may alternatively or additionally be used: the output of the machine learning model 106b may be used by a supervised training algorithm 212b, or a different supervised training algorithm 212a, to train the machine learning model 106a to identify anomalies identified by the machine learning model 106b.

In some implementations, training may proceed in various phases, each phase using one of the training approaches described above with respect to FIGS. 2A, 2B, and 2C. In a first example, machine learning models 106a, 106b are first trained using the supervised machine learning approach of FIG. 2A; the machine learning models 106a, 106b may then be trained using the unsupervised approach of FIG. 2B; and then the machine learning model 106b is further trained based on the output of the machine learning model 106a (and/or vice versa) according to the approach of FIG. 2C. In a second example, only unsupervised learning is used: the machine learning models 106a, 106b are individually trained using the unsupervised approach of FIG. 2B followed by further training machine learning model 106b based on the output of the machine learning model 106a and/or training the machine learning model 106a based on the output of the machine learning model 106b according to the approach of FIG. 2C.

FIG. 2C shows training with images 102, 104 with the understanding that one or more machine learning models for additional or alternative imaging modalities can be trained in the same manner. In particular, the output of a machine learning model according to one imaging modality may be used to train one or more other machine learning models according to one or more other imaging modalities in the same manner. The outputs of two or more first machine learning models for one or more first imaging modalities may be concatenated or otherwise combined and used to train one or more second machine learning models for one or more second machine learning models using the approach of FIG. 2C.

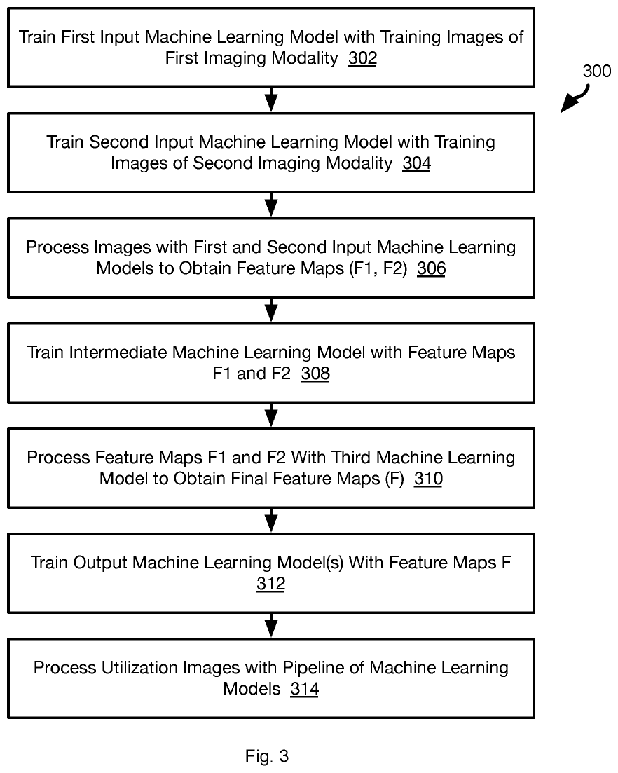

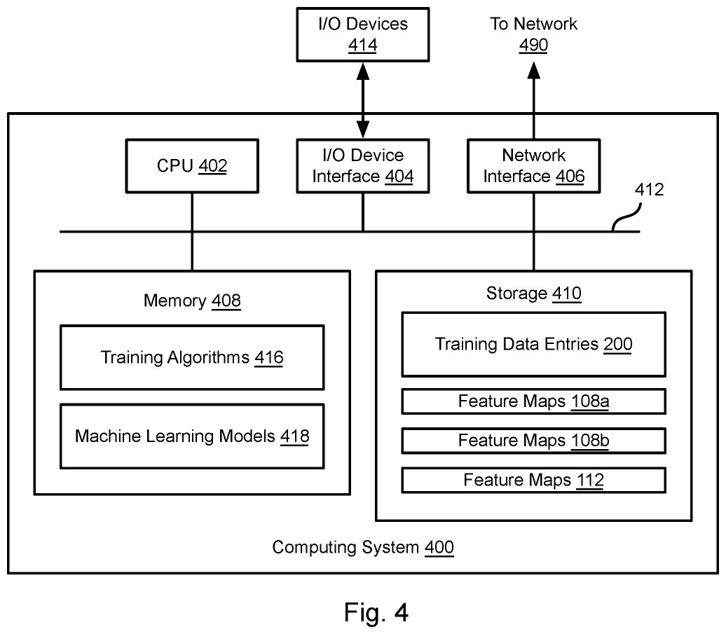

Referring to FIG. 3, the illustrated method 300 may be executed by a computer system, such as the computing system 400 of FIG. 4. The method 300 includes training, at step 302, a first input machine learning model with training images of a first imaging modality. For example, step 302 may include the machine learning model 106a with MCI images 102 according to any of the approaches described above with respect to FIGS. 2A to 2C.

The method 300 includes training, at step 304, a second input machine learning model with training images of a second imaging modality. For example, step 304 may include the machine learning model 106b with OCT images 104 according to any of the approaches described above with respect to FIGS. 2A to 2C.

The method 300 includes processing, at step 306, images according to the first imaging modality with the first input machine learning model to obtain input feature maps F1 and processing images according to the second imaging modality with the second input machine learning model to obtain input feature maps F2. The feature maps F1 and F2 may be outputs of hidden layers of the first and second input machine learning models, respectively. Step 306 may include processing MSI images 102 using the machine learning model 106a and processing OCT images 104 using the machine learning model 106b to obtain feature maps 108a, 108b as described above with respect to FIGS. 1 and 2A. As noted above, MSI images 102 and OCT images 104 may be part of a common training data entry 200 such that the MSI images 102 and OCT images 104 are of the same patient eye and captured substantially simultaneously.

The method 300 includes training, at step 308, an intermediate machine learning model with feature maps F1 and F2. Specifically, a plurality of pairs of feature maps F1 and F2 may each be processed by the intermediate machine learning model and the output of the intermediate machine learning model may be used to train the intermediate machine learning model. Each pair of feature maps F1 and F2 may be obtained for images of the first and second modality that are images of the same patient eye and captured substantially simultaneously. Step 308 may include processing the images used to obtain each pair of feature maps F1 and F2 using the intermediate machine learning model. Step 308 may include training a machine learning model 110 using feature maps 108a, 108b and training data entries 200 as described above with respect to FIG. 2A.

The method 300 includes processing, at step 310, feature pairs of feature maps F1 and F2, and possibly the training images used to obtain the feature maps F1 and F2 of each pair, with the intermediate machine learning model to obtain final feature maps F. The final feature maps F may be obtained from the output of a hidden layer of the intermediate machine learning model. Step 310 may include processing feature maps 108a, 108b, and possibly corresponding images 102, 104, using the machine learning model 110 to obtain feature maps 112 as described above with respect to FIGS. 1 and 2A.

The method 300 includes training, at step 312, one or more output machine learning models with the feature maps F. The one or more output machine learning models may be trained to output, for a given feature map F, an estimated representation of a pathology represented in the training images used to generate the feature map F using the first and second input machine learning models and the intermediate machine learning model. The one or more output machine learning models may take as an input the images according to the first and second imaging modalities that were used to generate the feature map F. Step 312 may include training one or both of machine learning models 114, 116 using the feature map 112 and possibly corresponding images 102, 104, to output some or all of a biomarker segmentation map 118, disease diagnosis 120, and a severity score 122.

The method 300 may include processing, at step 314, utilization images according to the first and second imaging modalities according to a pipeline of the first and second input machine learning models, the intermediate machine learning model, and the one or more output machine learning models. Specifically, one or more of the utilization images according to the first imaging modality are processed using the first input machine learning model to obtain a feature map F1; one or more of the utilization images according to the second imaging modality are processed using the second input machine learning model to obtain a feature map F2; the feature maps F1 and F2, and possibly the utilization images, are processed using the intermediate machine learning model to obtain a feature map F; and the feature map F, and possibly the utilization images, are processed by the one or more output machine learning models to obtain an estimated representation of a pathology represented in the utilization images. The estimated representation may be output to a display device or stored in a storage device for later usage or subsequent processing. Feature maps (F1, F2, F) may additionally be displayed or stored.

For example, step 314 may include processing utilization images 102, 104, i.e., images 102, 104 that are not part of a training data entry 200, using the machine learning models 106a, 106b, respectively, to obtain feature maps 108a, 108b, respectively, as described above with respect to FIG. 1. The feature maps 108a, 108b, and possibly the utilization images 102, 104, may be processed using the machine learning models 110 to obtain a feature map 112. The feature map 112, and possibly the utilization images 102, 104, may be processed by one or both of the machine learning models 114, 116 to obtain a biomarker segmentation map 118, disease diagnosis 120, and severity score 122.

The steps 302-314 may be performed in order, i.e. the first and second input machine learning models are trained, followed by training the intermediate machine learning model, followed by training the one or more output machine learning models, followed by utilization. Steps 302-314 may additionally or alternatively be interleaved, i.e., the first and second input machine learning models, the intermediate machine learning model, and the one or more output machine learning models being trained as a group. For example, in a first stage, the first and second input machine learning models, the intermediate machine learning model, and the one or more output machine learning models are trained separately in the order listed and, in a second stage, training continues as a group, i.e., subsequent to an iteration including processing a set of images according to the pipeline, some or all of the first and second input machine learning models, the intermediate machine learning model, and the one or more output machine learning models may be updated as part of the iteration by a training algorithm according to the outputs of the first and second input machine learning models, the intermediate machine learning model, and the one or more output machine learning models, respectively. Training individually or as a group may continue during the utilization step 314, particularly unsupervised learning as described with respect to FIGS. 2B and/or 2C.

Step 314 may be performed by a different computer system than is used to perform steps 302-312. For example, the pipeline including the first, second, third, and one or more output machine learning models may be installed on one or more other computer systems for use by surgeons or other health professionals.

Although the method 300 is described with respect to two imaging modalities, three or more imaging modalities may be used in a like manner. For example, suppose there are imaging modalities IMᵢ, i=1 to N, where N is greater than or equal to two. For a given training data entry, requirements of substantially identical scaling, alignment, and simultaneous imaging of the same eye of a patient may be met by images of the imaging modalities IMᵢ. There may be input machine learning models MLᵢ, i=1 to N, each machine learning model MLᵢ corresponding to an imaging modality and each generating a corresponding feature map Fᵢ by processing one or more images of the corresponding imaging modality IMᵢ. Each input machine learning model MLᵢ may be trained with images of the corresponding imaging modality Imᵢ according to any of the approaches described above for training the machine learning models 106a, 106b.

The intermediate machine learning model in such embodiments would therefore take N feature maps Fᵢ, i=1 to N, as inputs, and possibly the training images used to generate the feature maps Fᵢ. The output machine learning model would take as inputs the final feature map F and possibly the training images used to generate the feature maps Fᵢ. The intermediate machine learning model and output machine learning model are trained as described above with respect to the machine learning model 110 and the machine learning models 114, 116.

FIG. 4 illustrates an example computing system 400 that implements, at least partly, one or more functionalities described herein with respect to FIGS. 1 to 3. The computing system 400 may be integrated with an imaging device capturing images according to one or more of the imaging modalities described herein or may be a separate computing device.

As shown, computing system 400 includes a central processing unit (CPU) 402, one or more I/O device interfaces 404, which may allow for the connection of various I/O devices 414 (e.g., keyboards, displays, mouse devices, pen input, etc.) to computing system 400, network interface 406 through which computing system 400 is connected to network 490, a memory 408, storage 410, and an interconnect 412.

In cases where computing system 400 is an imaging system, such the SLO, an OCT, or fundus camera, the computing system 400 may further include one or more optical components for obtaining ophthalmic imaging of a patient's eye as well as any other components known to one of ordinary skill in the art.

CPU 402 may retrieve and execute programming instructions stored in the memory 408. Similarly, CPU 402 may retrieve and store application data residing in the memory 408. The interconnect 412 transmits programming instructions and application data, among CPU 402, I/O device interface 404, network interface 406, memory 408, and storage 410. CPU 402 is included to be representative of a single CPU, multiple CPUs, a single CPU having multiple processing cores, and the like.

Memory 408 is representative of a volatile memory, such as a random access memory, and/or a nonvolatile memory, such as nonvolatile random access memory, phase change random access memory, or the like. As shown, memory 408 may store training algorithms 416, such as any of the training algorithms 202, 204, 206a, 206b, 210a, 210b, 212a, 212b described herein. The memory 408 may further store machine learning models 418, such as any of the machine learning models 106a, 106b, 110, 114, 116 described herein.

Storage 410 may be non-volatile memory, such as a disk drive, solid state drive, or a collection of storage devices distributed across multiple storage systems. Storage 410 may optionally store training data entries 200 or other collections of MSI images 102 and OCT images 104 for training and/or utilization according to the system and method described herein. Storage 410 may optionally store intermediate results of processing by any of the machine learning models 106a, 106b, 110, 114, 116, such as feature maps 108a, 108b, 112.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module. The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
one or more processing devices and one or more memory devices coupled to the one or more processing devices, the one or more memory devices storing executable code that, when executed by the one or more processing devices, causes the one or more processing devices to:
for each imaging modality of a plurality of imaging modalities:
process one or more images according to each imaging modality using an input machine learning model of a plurality of input machine learning models corresponding to each imaging modality to obtain an input feature map, the one or more images being images of an eye of a patient;
process the input feature maps for the plurality of imaging modalities using an intermediate machine learning model to obtain a final feature map; and
process the final feature map using one or more output machine learning models to obtain one or more estimated representations of a pathology of the eye of the patient, the one or more estimated representations of the pathology of the eye of the patient comprising a diagnosis of a retinal tear and a severity score for the diagnosis.

2. A system comprising:
one or more processing devices and one or more memory devices coupled to the one or more processing devices, the one or more memory devices storing executable code that, when executed by the one or more processing devices, causes the one or more processing devices to:
for each imaging modality of a plurality of imaging modalities:
process one or more images according to each imaging modality using an input machine learning model of a plurality of input machine learning models corresponding to each imaging modality to obtain an input feature map, the one or more images being images of an eye of a patient;
process the input feature maps for the plurality of imaging modalities using an intermediate machine learning model to obtain a final feature map; and
process the final feature map using one or more output machine learning models to obtain one or more estimated representations of a pathology of the eye of the patient.

3. The system of claim 2, wherein the plurality of imaging modalities include at least one of multispectral imaging (MSI) or optical coherence tomography (OCT).

4. The system of claim 2, wherein the plurality of imaging modalities include multispectral imaging (MSI) and optical coherence tomography (OCT).

5. The system of claim 2, wherein each input machine learning model is one of a neural network, a deep neural network (DNN), a convolution neural network (CNN), a recurrent neural network (RNN), a region-based CNN (R-CNN), and an autoencoder (AE).

6. The system of claim 2, wherein the input feature map for each imaging modality is an output of a hidden layer of the input machine learning model for each imaging modality.

7. The system of claim 2, wherein the intermediate machine learning model is one of a neural network, a deep neural network (DNN), a convolution neural network (CNN), a recurrent neural network (RNN), a region-based CNN (R-CNN), and an autoencoder (AE).

8. The system of claim 2, wherein the final feature map is an output of a hidden layer of the intermediate machine learning model.

9. The system of claim 2, wherein the one or more output machine learning models are one of a neural network, a deep neural network (DNN), a convolution neural network (CNN), a recurrent neural network (RNN), a region-based CNN (R-CNN), an autoencoder (AE), a long short term memory (LSTM) machine learning model, and a generative adversarial network (GAN) machine learning model.

10. The system of claim 2, wherein the one or more estimated representations of the pathology of the eye of the patient comprises a diagnosis of the pathology.

11. The system of claim 10, wherein the one or more estimated representations of the pathology of the eye of the patient comprises a severity score for the diagnosis.

12. The system of claim 2, wherein the one or more estimated representations of the pathology of the eye of the patient comprise one or more biomarker segmentation maps.

13. A method comprising:
for each imaging modality of a plurality of imaging modalities:
processing, by a computer system, one or more images according to each imaging modality using an input machine learning model of a plurality of input machine learning models corresponding to each imaging modality to obtain an input feature map, the one or more images being images of an eye of a patient;
processing, by the computer system, the input feature maps for the plurality of imaging modalities using an intermediate machine learning model to obtain a final feature map; and
processing, by the computer system, the final feature map using one or more output machine learning models to obtain one or more estimated representations of a pathology of the eye of the patient.

14. The method of claim 13, wherein the plurality of imaging modalities include multispectral imaging (MSI) and optical coherence tomography (OCT).

15. The method of claim 13, wherein each input machine learning model and the intermediate machine learning model is one of a neural network, a deep neural network (DNN), a convolution neural network (CNN), a recurrent neural network (RNN), a region-based CNN (R-CNN), and an autoencoder (AE).

16. The method of claim 13, wherein the input feature map for each imaging modality is an output of a hidden layer of the input machine learning model for each imaging modality and the final feature map is an output of a hidden layer of the intermediate machine learning model.

17. The method of claim 13, wherein the one or more output machine learning models are one of a neural network, a deep neural network (DNN), a convolution neural network (CNN), a recurrent neural network (RNN), a region-based CNN (R-CNN), an autoencoder (AE), a long short term memory (LSTM) machine learning model, and a generative adversarial network (GAN) machine learning model.

18. The method of claim 13, wherein the one or more estimated representations of the pathology of the eye of the patient comprises a diagnosis of the pathology and a severity score for the diagnosis.

19. The method of claim 13, wherein the one or more estimated representations of the pathology of the eye of the patient comprise one or more biomarker segmentation maps.

20. The method of claim 13, wherein the pathology of the eye includes at least one of:
Retinal tear(s)
Retinal detachment
Diabetic retinopathy
Hypertensive retinopathy
Sickle cell retinopathy
Central retinal vein occlusion
Epiretinal membrane
Macular hole(s)
Macular degeneration (including age-related Macular Degeneration)
Retinal pigmentosa
Glaucoma
Alzheimer's disease
Parkinson's disease.
